(19) United States
(12) Patent Application Publication
(10) Pub. No.: US 2010/0125421 A1
(43) Pub. Date: May 20, 2010

(54) System and method for determining a dosage for a treatment

(76) Inventor: Howard Jay Snortland, Menlo Park, CA (US)

Correspondence Address: KOLISCH HARTWELL, P.C., 200 PACIFIC BUILDING, 520 SW YAMHILL STREET, PORTLAND, OR, 97204, US

(21) Appl. No.: 12/291,939

(22) Filed: Nov. 14, 2008

(51) Int. Cl. G06F 17/30; G06F 17/00; G06F 15/16; G06Q 50/00; C12Q 1/68

(52) U.S. Cl. 702/20; 715/234; 709/219; 705/3; 435/6; 380/28; 707/E17.002; 707/E17.044; 707/608

(57) ABSTRACT

An system and method for allowing the real-time diagnostics of various genotype-related treatments while allowing for the changing of demographic data such as a person's age, weight, etc. Various embodiments and methods of new processes include the assembly and association of genetic material samples, the preparation of microarrays with representative genetic material samples in a pattern best suited for analysis as well as manipulation, and delivery of assimilated and compiled data in the form of an electronic document for determining a dosage for a treatment.

### CROSS-REFERENCE TO RELATED PATENT APPLICATION

This patent application is related to the patent application entitled “AN ELECTRONIC DOCUMENT FOR AUTOMATICALLY DETERMINING A DOSAGE FOR A TREATMENT” filed concurrently on Nov. 14, 2008.

### BACKGROUND

The advance of genetics has led to breakthroughs in clinical diagnostics allowing physicians to more properly diagnose symptoms that lead to the prescription of a dosage for a treatment. Routine treatments for various conditions can be better prescribed when the physician knows specific genetic markers within the patient that the physician is treating. As a result, certain diseases and developed conditions may be addressed in a more efficient manner using genetics.

Furthermore, genetic disorders afflict many people and remain the subject of much study and misunderstanding. Typical genetic disorders occur when specific gene sequences are not maintained as expected, such as with Phenylketonuria and Xeroderma pigmentosum. Currently, around 4,000 genetic disorders are known, with more being discovered as more is understood about the human genome. Most disorders are quite rare and affect one person in every several thousands or millions while other are more common, such as cystic fibrosis wherein about 5% of the population of the United States carries at least one copy of the defective gene.

A person's genetic makeup is reflected through Deoxyribonucleic Acids (DNA). DNA is a molecule that comprises sequences of nucleic acids (i.e., nucleotides) that form the code which contains the genetic instructions for the development and functioning of living organisms. A DNA sequence or genetic sequence is a succession of any of four specific nucleic acids representing the primary structure of a real or hypothetical DNA molecule or strand, with the capacity to carry information. As is well understood in the art, the possible nucleic acids (letters) are A, C, G, and T, representing the four nucleotide subunits of a DNA strand—adenine, cytosine, guanine, and thymine bases covalently linked to phospho-backbone. Typically the sequences are printed abutting one another without gaps, as in the sequence AAAGTCTGAC. A succession of any number of nucleotides greater than four may be called a sequence.

Ribonucleic acid (RNA) is a nucleic acid polymer consisting of nucleotide monomers, that acts as a messenger between DNA and ribosomes, and that is also responsible for making proteins by coding for amino acids. RNA polynucleotides contain ribose sugars unlike DNA, which contains deoxyribose. RNA is transcribed (synthesized) from DNA by enzymes called RNA polymerases and further processed by other enzymes. RNA serves as the template for translation of genes into proteins, transferring amino acids to the ribosome to form proteins, and also translating the transcript into proteins.

A gene is a segment of nucleic acid that contains the information necessary to produce a functional product, usually a protein. Genes contain regulatory regions dictating under what conditions the product is produced, transcribed regions dictating the structure of the product, and/or other functional sequence regions. Genes interact with each other to influence physical development and behavior. Genes consist of a long strand of DNA (RNA in some viruses) that contains a promoter, which controls the activity of a gene, and a coding sequence, which determines what the gene produces. When a gene is active, the coding sequence is copied in a process called transcription, producing an RNA copy of the gene's information. This RNA can then direct the synthesis of proteins via the genetic code. However, RNAs can also be used directly, for example as part of the ribosome. These molecules resulting from gene expression, whether RNA or protein, are known as gene products.

The total complement of genes in an organism or cell is known as its genome. The genome size of an organism is loosely dependent on its complexity. The number of genes in the human genome is estimated to be just under 3 billion base pairs and about 30,000 genes.

As previously mentioned, certain genetic mutations and/or disorders may result from DNA sequences being incorrectly coded. A Single Nucleotide Polymorphism or SNP (often times called a “snip”) is a DNA sequence variation occurring when a single nucleotide—A, T, C, or G—in the genome (or other shared sequence) differs between members of a species (or between paired chromosomes in an individual). For example, two sequenced DNA fragments from different individuals, AAGCCTA to AAGCTTA, contain a difference in a single nucleotide. In this case, this situation may be referred to as having two alleles: C and T. Most common SNPs possess only 2 alleles. Generally speaking for a variation to be considered a SNP, as opposed to a spontaneous point mutation, a variation must appear in at least 1% of the population.

Within a population, Single Nucleotide Polymorphisms can be assigned a minor allele frequency—the ratio of chromosomes in the population carrying the less common variant to those with the more common variant. It is important to note that there are variations between human populations, so a Single Nucleotide Polymorphism that is common enough for inclusion in one geographical or ethnic group may be much rarer in another. As of 2007, there are approximately 10⁷ SNPs known in humans.

Single Nucleotide Polymorphisms may fall within coding sequences of genes, noncoding regions of genes, or in the intergenic regions between genes. Single Nucleotide Polymorphisms within a coding sequence will not necessarily change the amino acid sequence of the protein that is produced, due to degeneracy of the genetic code. A Single Nucleotide Polymorphism in which both forms lead to the same polypeptide sequence is termed synonymous (sometimes called a silent mutation)—if a different polypeptide sequence is produced they are non-synonymous. Single Nucleotide Polymorphisms that are not in protein coding regions may still have consequences for gene splicing, transcription factor binding, or the sequence of non-coding RNA.

Variations in the DNA sequences of humans can affect how humans develop diseases, and/or respond to pathogens, chemicals, drugs, etc. However, one aspect of learning about DNA sequences that is of great importance in biomedical research is comparing regions of the genome between people (e.g., comparing DNA sequences from similar people, one with a genetic mutation and one without the genetic mutation). Technologies from Affymetrix™ and Illumina™ (for example) allow for genotyping hundreds of thousands of Single Nucleotide Polymorphisms for typically under $1,000.00 in a couple of days.

Microarray analysis techniques are typically used in interpreting the data generated from experiments on DNA, RNA, and protein microarrays, which allow researchers to investigate the expression state of a large number of genes—in many cases, an organism's entire genome—in a single experiment. Such experiments generate a very large volume of genetic data that can be difficult to analyze, especially in the absence of good gene annotation. Most microarray manufacturers, such as Affymetrix™, provide commercial data analysis software with microarray equipment such as plate readers.

Specialized software tools for statistical analysis to determine the extent of over- or under-expression of a gene in a microarray experiment relative to a reference state have also been developed to aid in identifying genes or gene sets associated with particular phenotypes. Examples of the former include GeneSpring GX and of the latter GeneSpring GT, both available from Agilent Technologies, Inc. Such statistics packages typically offer the user information on the genes or gene sets of interest, including links to entries in databases such as NCBI's GenBank and curated databases such as Biocarta and Gene Ontology.

As a result of a statistical analysis, specific aspects of an organism may be genotyped. Genotyping refers to the process of determining the genotype of an individual with a biological assay. Current methods of doing this include Polymerase Chain Reaction (PCR), DNA sequencing, and hybridization to DNA microarrays or beads.

Further, phenotyping is also a known process for assessing phenotypes. The phenotype of an individual organism is either its total physical appearance and constitution or a specific manifestation of a trait, such as size, eye color, or behavior that varies between individuals. Phenotype is determined to a large extent by genotype, or by the identity of the alleles that an individual carries at one or more positions on the chromosomes. Many phenotypes are determined by multiple genes and influenced by environmental factors. Thus, the identity of one or a few known alleles does not always enable prediction of the phenotype. The proportion of a group of individuals bearing a particular allele that also possess a phenotype that expresses that allele is known as an allele's penetrance.

With the context of knowing an individual's specific genetic makeup through genetic sampling and analysis, certain diagnostics may be more accurately assessed. In one example, Warfarin dosage may be more accurately determined through a genetic assessment of the presence, or lack thereof, of known gene sequences.

Warfarin (also known under the brand names of Coumadin, Jantoven, Marevan, and Waran) is an anticoagulant medication that is administered to assist with preventing clotting of blood. In its medical use, Warfarin is used for the prophylaxis of thrombosis and thromboembolism in many disorders or in post-surgical situations. Compared with other pharmaceuticals, Warfarin is considered to have a narrow “therapeutic window”, meaning the minimum dose needed to achieve a useful, therapeutic effect does not differ greatly from the maximum safe dose above which adverse effects such as uncontrolled bleeding may occur. In addition, the correct dosage of Warfarin as a treatment varies from person to person and is based upon a number of physical and genetic characteristics.

As is the case for Warfarin, sometimes treatments may be better diagnosed using genetic analysis. As such, through genetic analysis, the presence or lack of presence of known gene variants helps determine dosages for some treatments. An analyte is a substance or chemical constituent that is determined in an analytical procedure, such as a titration. In this context, an analyte refers to a particular allele whose presence or absence in a patient's genome is to be determined by a genetic test.

In the past, Warfarin dosage was determined by a physician using an educated guess to begin a series of “trial and error” dosages. As the physician administered specific dosages, the dosage could be increased or decreased based upon the change in condition of the patient. With the advent of more prevalent genetic diagnostics, physicians could then rely on a more accurate algorithm for determining a dosage based upon demographic input and genetic information gleaned from the patient.

In a common practice, a physician would obtain a genetic sample of a patient and send the genetic sample along with specific demographic data (e.g., height, weight, ethnicity) to a diagnostics facility that would analyze the sample for the presence of known gene sequences. The facility would then generate a dosage report that was based on the genetic markers found and the given demographic data. The dosage report could then be faxed or mailed to the physician.

However, existing testing and delivery methods for genotyping result in a diagnostic that is static in time. That is, when a dosage is determined through a complex algorithm that takes into account not only the essentially unchangeable genetic information, but also other demographic information, (such as age, weight, present smoker), the dosage determined is unique to that set of demographic details at that moment in time. A year later, the patient may weigh less, be one year older or have ceased smoking resulting in different demographic data. Thus, the previous dosage report is no longer correct and the diagnostic must be repeated. Since physicians typically do not waste time learning and knowing the complex algorithms used to determine such dosages, the entire test is often repeated.

Some newer solutions have been implemented including using a website to provide an interface for physician's to input genetic and demographic data to return a dosage recommendation. However, these real-time web solutions provide little or no security (especially in light of the Health Insurance Portability and Accountability Act (HIPAA) in the United States) and rely on accurate keyed entry of complex genetic data. Such time-consuming re-entry of data is prone to human error, problematic and unreliable.

What is needed is a more secure and repeatable method for implementing complex algorithms for determining a dosage of a treatment based upon genetic and demographic data that may be dynamic in nature.

### BRIEF DESCRIPTION OF THE DRAWINGS

The foregoing aspects and many of the attendant advantages of the claims will become more readily appreciated as the same become better understood by reference to the following detailed description, when taken in conjunction with the accompanying drawings, wherein:

FIG. 1 shows a diagram of a method for preparing a microarray to be used in a broad-based gene transcript test according to an embodiment of an invention disclosed herein;

FIG. 2 is a diagrammatic representation of a suitable computing environment in which some aspects of a broad-based gene-transcript test may be used to generate an electronic document for determining a dosage for a treatment according to an embodiment of an invention disclosed herein;

FIG. 3 is a diagrammatic representation of a system and method for establishing a data structure to be used to generate an electronic document for determining a dosage for a treatment from a broad-based gene transcript test according to an embodiment of an invention disclosed herein;

FIG. 4 is an electronic document showing genetic information and demographic information about a patient according to an embodiment of an invention disclosed herein;

FIG. 5 is a flowchart of an overall method for generating an electronic document for determining a dosage for a treatment according to an embodiment of an invention disclosed herein;

FIG. 6 is a flowchart of a particular method for realizing an electronic document for determining a dosage for a treatment according to an embodiment of an invention disclosed herein;

FIG. 7 is a diagram of a system for testing an electronic document for determining a dosage for a treatment as generated by the method of FIGS. 5 and 6 according to an embodiment of an invention disclosed herein; and

FIG. 8 is a flowchart of a method for diagnosing a patient for a dosage of a treatment using an electronic document having the patient's genetic information and demographic information according to an embodiment of an invention disclosed herein.

### DETAILED DESCRIPTION

The following discussion is presented to enable a person skilled in the art to make and use the subject matter disclosed herein. The general principles described herein may be applied to embodiments and applications other than those detailed above without departing from the spirit and scope of the present detailed description. The present disclosure is not intended to be limited to the embodiments shown, but is to be accorded the widest scope consistent with the principles and features disclosed or suggested herein.

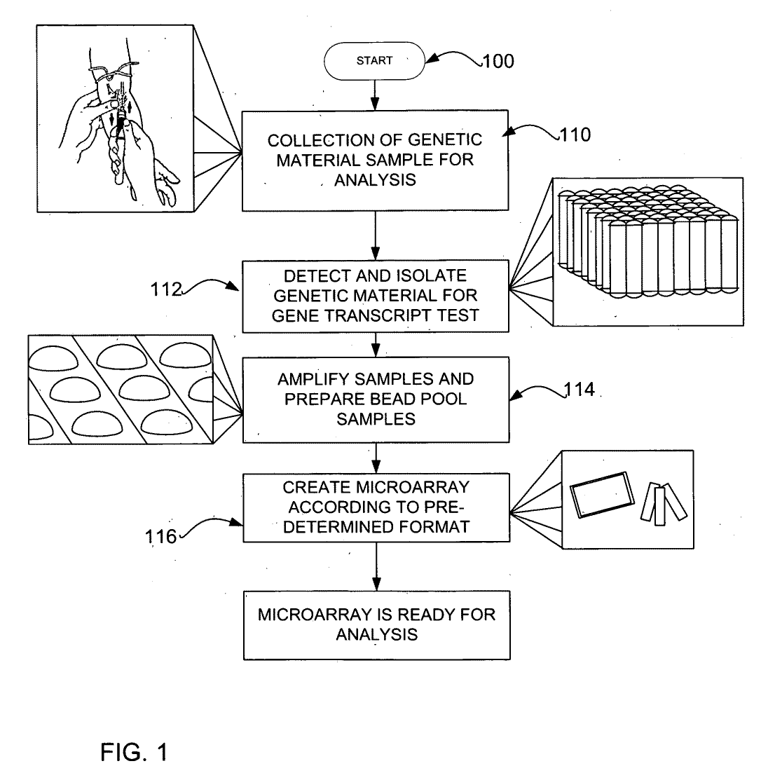

The subject matter disclosed herein is related to a system and method for using an electronic document suitable for allowing the real-time diagnostics of various genotype-related treatments while allowing for the changing of demographic data such as a person's age, weight, etc. Various embodiments and methods of new processes include the assembly and association of genetic material samples, the preparation of microarrays with representative genetic material samples in a pattern best suited for analysis as well as manipulation, and delivery of assimilated and compiled data in the form of an electronic document for determining a dosage for a treatment. Various aspects of these embodiments are discussed in FIGS. 1-8 below.

FIG. 1 shows a diagram of an overall method 100 for preparing genetic samples that may be used in a broad-based gene transcript test according to an embodiment of an invention disclosed herein. The method may typically include drawing a blood sample (or obtaining another source of genetic material) from a patient scheduled for genotyping in step 110. It should be noted that a wide variety of biological materials may be used as a source of genetic material (e.g., DNA and/or RNA),including but not limited to blood, saliva, urine, tissue samples or cervical scrapings. Blood cells are easily collected and easily transported making this source for DNA/RNA efficient and effective. The blood sample may typically be collected using a suitable blood collection device such as blood collection tubes that are available from Paxgene™.

The sample is typically properly tagged and labeled by an anonymous yet traceable patient identification, i.e., abstracted from the patient. That is, all measures are taken to comply with the Health Insurance Portability and Accountability Act (HIPAA) such that the blood sample is identifiable but also protected from accidental disclosure of privileged information. At the time of collection, additional demographic information may be stored (e.g., written on a tag, stored in a computer database) with the blood sample. Such demographic information may include a number of different patient characteristics and descriptions, such as age, sex, country of origin, race, specific health issues, occupation, birthplace, current living location, etc.

Specific genetic material, such as RNA from the blood sample, may then be detected and isolated in step 112 using an RNA isolation kit such as those that are available from Qiagen™. As mentioned above, RNA isolation may be accomplished at the same physical location as collection or may be accomplished at a remote laboratory after collection.

At step 114, specific sequences in an RNA sample may be amplified using a fluorescence process that may be specific to pre-determined strands of RNA such as available from Illumina™ in a product entitled DASL™. In an alternative embodiment, specific sequences in DNA may also be amplified using a similar fluorescence process that may be specific to pre-determined strands of DNA such as available from Illumina™ in a product entitled Golden Gate™.

The isolation of genetic materials is typically followed by amplification of fluorescently labeled copies that may then be hybridized to specific probes attached to a common substrate, i.e., a microarray. However, the collected and isolated samples may be arranged and analyzed in any manner suitable for analysis.

At step 116, the isolated and amplified samples of genetic material may be grouped according to identified sets of strands of genetic material. The groups may be arranged in a specific pattern in bead pools on a microarray according to a predetermined format. Such predetermined formats may include a standard format suitable for individual analysis of all identified genes in isolated RNA/DNA strands. Other predetermined formats may include a side-by-side comparison to one or more control groups of similar genes from control group samples. Other formats may include specific sets of genes suitable for broad-based genetic mutation association, multiple sclerosis association, broad-based diagnostics collection, broad-based predictive treatment data sets, or any other association of genes with samples. Once the microarray has been created in a specific pattern, the emergence of patterns and the like may be ready for analysis at step 118. The preparation of such a microarray is described in more detail in U.S. patent application Ser. No. 11/775,660 entitled, “Method and System for Preparing a Microarray for a Disease Association Gene Transcript Test,” assigned to IGD-Intel of Seattle, Wash., which is incorporated by reference. The formats for arranging samples in a microarray typically follow specifics associated with the groupings of blood samples. With such a genetic sample prepared for analysis, any number of analytic tests may be performed to determine the presence of known gene markers. This analytic data may then be stored in a database as described further below.

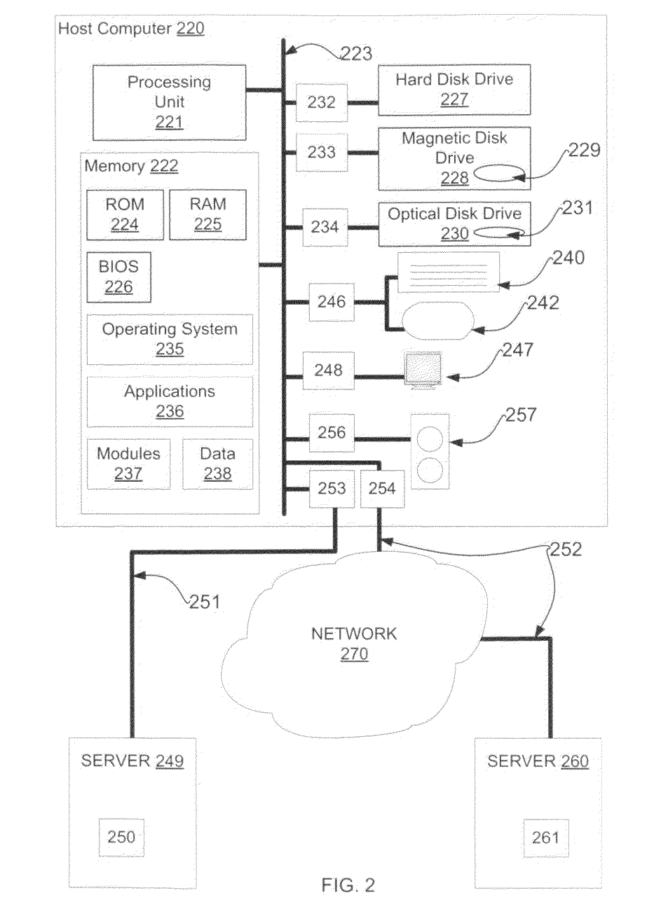

FIG. 2 is a diagrammatic representation of a suitable computing environment in which some aspects of a broad-based gene-transcript test may be practiced according to an embodiment of an invention disclosed herein. With reference to FIG. 2, an exemplary system for implementing the invention includes a general purpose computing device in the form of a conventional personal computer 220, (sometimes called a host computer or client computer) including a processing unit 221, a system memory 222, and a system bus 223 that couples various system components including the system memory 222 to the processing unit 221. The system bus 223 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

The system memory 222 includes Read Only Memory (ROM) or Electrically Erasable Programmable Read Only Memory (EEPROM) 224 and random access memory (RAM) 225. A basic input/output system (BIOS) 226, containing the basic routines that help to transfer information between elements within the personal computer 220, such as during start-up, is stored in ROM or EEPROM 224. The personal computer 220 further includes a hard disk drive 227 for reading from and writing to a hard disk, not shown, a magnetic disk drive 228 for reading from or writing to a removable magnetic disk 229, and an optical disk drive 230 for reading from or writing to a removable optical disk 231 such as a CD ROM or other optical media. The hard disk drive 227, magnetic disk drive 228, and optical disk drive 230 are connected to the system bus 223 by a hard disk drive interface 232, a magnetic disk drive interface 233, and an optical drive interface 234, respectively. The drives and their associated computer-readable media provide nonvolatile storage of computer readable instructions, data structures, program modules and other data for the personal computer 220. Although the exemplary environment described herein employs a hard disk, a removable magnetic disk 229 and a removable optical disk 231, it should be appreciated by those skilled in the art that other types of computer-readable media which can store data that is accessible by a computer, such as portable thumb drives, magnetic cassettes, flash memory cards, digital versatile disks, Bernoulli cartridges, random access memories (RAMs), read only memories (ROM), and the like, may also be used in the exemplary operating environment.

A number of program modules may be stored on the hard disk, magnetic disk 229, optical disk 231, ROM or EEPROM 224 or RAM 225, including an operating system 235, one or more application programs 236, other program modules 237, and program data 238. A user may enter commands and information into the personal computer 220 through input devices such as a keyboard 240 and pointing device 242. Other input devices (not shown) may include a microphone, joystick, game pad, satellite dish, scanner, or the like. These and other input devices are often connected to the processing unit 221 through one or more serial port interfaces 246 that are coupled to the system bus 223, but may be connected by other interfaces, such as a parallel port, game port or a universal serial bus (USB). A monitor 247 or other type of display device is also connected to the system bus 223 via an interface, such as a video adapter 248. One or more speakers 257 are also connected to the system bus 223 via an interface, such as an audio adapter 256. In addition to the monitor and speakers, personal computers typically include other peripheral output devices (not shown), such as printers.

The personal computer 220 typically operates in a networked environment using logical connections to one or more remote computers, such as remote computers 249 and 260. Each remote computer 249 or 260 may be another personal computer, a server, a router, a network PC, a peer device or other common network node, and typically includes many or all of the elements described above relative to the personal computer 220, although only a memory storage device 250 or 261 has been illustrated in FIG. 2. The logical connections depicted in FIG. 2 include a local area network (LAN) 251 and a wide area network (WAN) 252. Such networking environments are commonplace in offices, enterprise-wide computer networks, intranets and the Internet. As depicted in FIG. 2, the remote computer 260 communicates with the personal computer 220 via the local area network 251. The remote computer 249 communicates with the personal computer 220 via the wide area network 252.

When used in a LAN networking environment, the personal computer 220 is connected to the local network 251 through a network interface or adapter 253. When used in a WAN networking environment, the personal computer 220 typically includes a modem 254 or other means for establishing communications over the wide area network 252, such as the Internet. The modem 254, which may be internal or external, is connected to the system bus 223 via a serial port interface. In a networked environment, program modules depicted relative to the personal computer 220, or portions thereof, may be stored in the remote memory storage device. It will be appreciated that the network connections shown are exemplary and other means of establishing a communications link between the computers may be used.

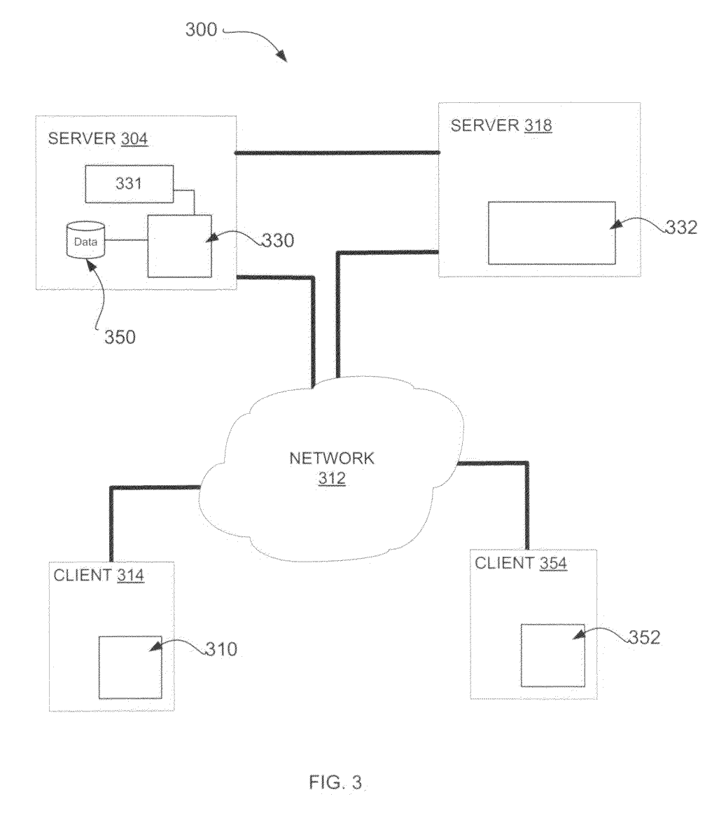

FIG. 3 is a diagrammatic representation of a system and method for establishing a data structure to be used to generate an electronic document for determining a dosage for a treatment from a broad-based gene transcript test according to an embodiment of an invention disclosed herein. The system 300 may typically include a number of interconnected computers. Such interconnected computers may include client computers 314 and 354 that are similar in nature to the personal computer 220 of FIG. 2. Each client computer 314 and 352 may be interconnected to each other via a network 312. Such a network 312 may be a local area network, such as within a building or may be a wide-area network, such as the Internet.

The client computers 314 and 354 may also be communicatively coupled to one or more server computers 304 and 332. Delving further into the server computer 304, FIG. 3 shows a controller 331 coupled to the program module 331 as well as a database 350. In this example, the database 350 may contain abstracted patient and physician data as detailed further below while the program module 330 may be associated with a broad-based gene transcript test.

Such a computer network 300 may be used to facilitate genetic diagnostics to assist physicians in determining a proper dosage for a treatment. For example, one method utilizes communication mediums to transfer electronic documents from various computer platforms to another. In one embodiment, a system for calculating a dosage of a treatment using a personalized diagnostic electronic document, may be realized within the computer network 300. A genetic sample collection system 352 operable to collect a sample of genetic material from a patient may reside within a facility utilizing a first client computer 354. Further, a genetic sample analysis system 332 operable to receive the sample of genetic material and operable to identify the presence of at least one of a plurality of gene markers may reside at a facility associated with the server computer 318. Further yet, a client computer 314 at a third location may be operable to generate an electronic document 310 having collected genetic data embedded therein. Finally, the collected genetic data may also be further analyzed at another server computer 304 using a program module 330 for a broad-based gene-transcript test facilitated by a local controller 331 and stored in a database that includes abstracted identification information.

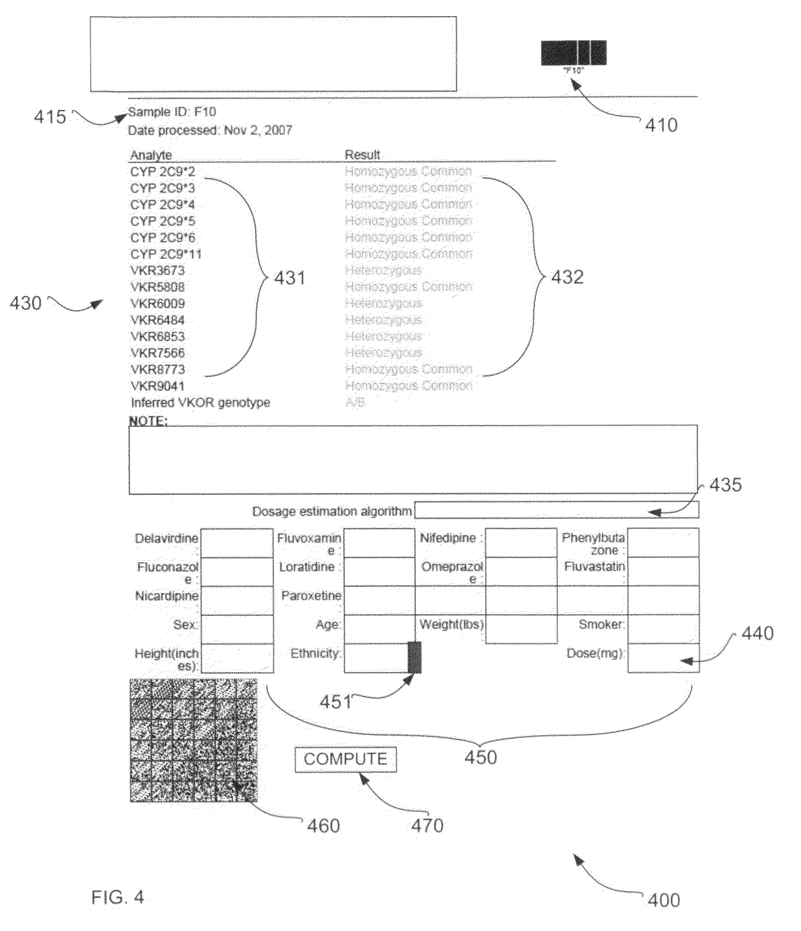

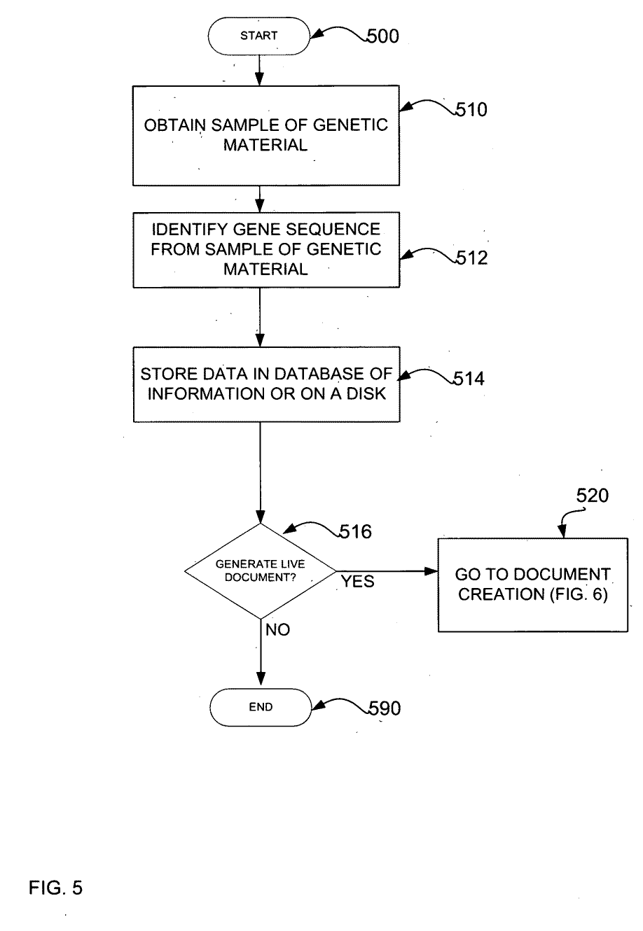

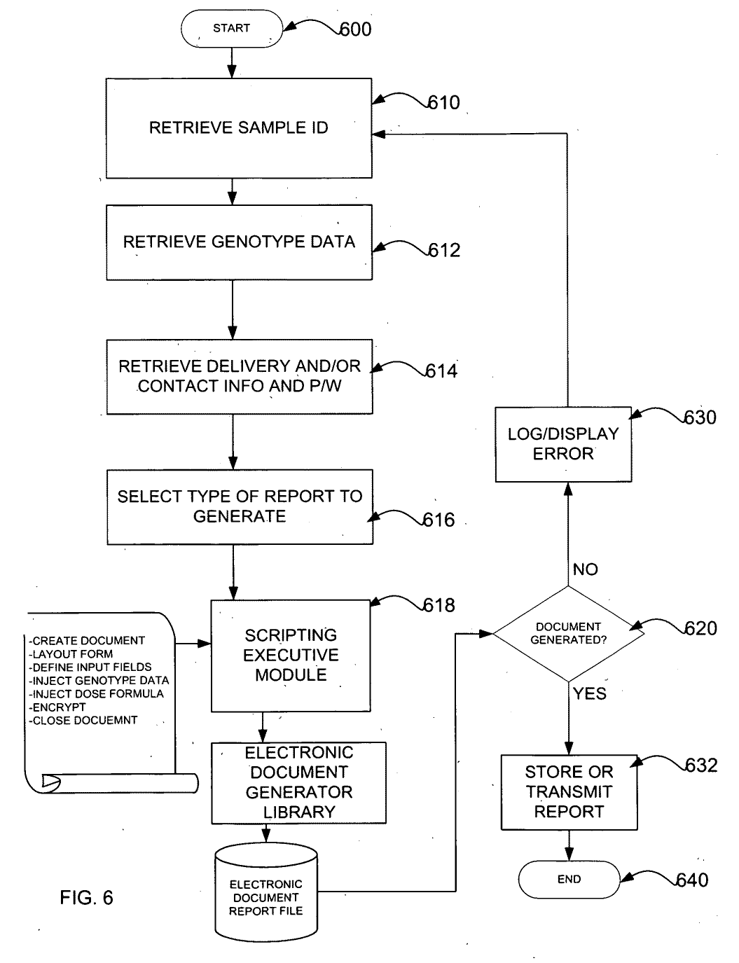

Those skilled in the art will appreciate that the above example of the delineations between computers is exemplary and any number of variations may be introduced and permutations to the architecture of the computer network 300 may be implemented yielding the same functionality and utility. As such, an electronic document, as described below with respect to FIG. 4 may be generated and utilized in several ways within the computer network 300. Various methods may be practiced in this computing environment as detailed below with respect to FIGS. 5 and 6.

FIG. 4 is an electronic document 400 showing genetic information 430 and demographic information about a patient according to an embodiment of an invention disclosed herein. By way of overview, this electronic document 400 comprises embedded data representing genetic information 430 corresponding to a genetic sample from a living being. The electronic document 400 further comprises input fields 450 operable to receive an input corresponding to demographic data about the living being. Finally, the electronic document 400 comprises a computed field 440 to display the dosage when the compute button 470 is clicked to calculate a dosage of a treatment based on the embedded genetic data 430 and the inputted demographic data.

Looking into these aspects in more detail, the embedded genetic data 430 typically corresponds to a human being (although the underlying organism may be any living being) and the presence of at least one known single nucleotide polymorphism or to the presence of at least one known gene sequence or gene marker within the human genomic data. In this electronic document 400, an analyte 431 is identified in a first column and result 432 is displayed in a second column. This genetic information is displayed for informational purposes only and cannot be changed without having authorship credentials over the electronic document 400. That is, the genetic information 430 is embedded for the sake of preventing accidental data over-writes or changes. Of course, once a patient's genetic information is determined and embedded, there is no need to change the data, as a patient's genomic characteristics do not appreciably change over time.

Typically, the list of analytes is dictated by the underlying treatment in which a physician seeks diagnostics for a dosage. In this example, a Warfarin dosage is sought and the list of analytes 431 corresponds to gene markers used in calculating a dosage for a Warfarin treatment. Thus, the list of analytes may typically include at least one of: CYP 2C9*2, CYP 2C9*3, CYP 2C9*4, CYP 2C9*5, CYP 2C9*6, CYP 2C9*11, VKR3673, VKR5808, VKR6009, VKR6484, VKR6853, VKR7566, VKR8773, and VKR9041.

With embedded genetic data 430 in place, the electronic document may also include a plurality of input fields 450 for demographic data about the living being. Typical input fields may include demographics data such as age, weight, height, gender, ethnicity, propensity to smoke, and genotype data. Further, the demographic data may also indicate information in the way of medical history, such as use of delavirdine, use of fluvoxamine, use of nifedipine, use of phenylbutozone, use of fluconazole, use of loratodine, use of omeprazol, use of fluvastatin, use of nicardipine, and use of paroxetine. Together with the embedded genetic data 430, various known algorithms may utilize some or all of this demographic data from the input fields 450 to determine a dosage for a treatment in a computational field 440.

Each input field may comprise a field wherein information may be typed via textual input from a keyed input device. However, other manners of data entry may also be realized, such as voice recognition, data retrieval from a remote database, retina scan database retrieval, thumb-print scan database retrieval, and the like. Further, some input fields 450 may include drop-down actuation buttons 451 and 435 that reveal lists of selectable choices in the field. For example, an ethnicity input field may be limited to the choices of White, Black, Asian, and Other. Further yet, some input fields may be associated with parameters, such that data entry outside the parameters are rejected. For example, weight may be a field requiring a numeric entry in kilograms such that an entry of alpha-characters or an entry of an obviously erroneous number (e.g., a patient who weighs 200,000 kg) would be rejected.

The computed field 440 may be associated with a known algorithm for calculating a dosage for a treatment. Using the running example, a more accurate dosage of the drug, Warfarin is realized when genetic information about the patient is known. In some embodiments, the algorithm for calculating the result is fixed and cannot be changed. In other embodiments, a dosage estimation algorithm field 435 may allow a user of the electronic document 400 to select from a group of potential algorithms. For example, in Warfarin dosage estimation, there exist a number of established and respected algorithms, including a University of Newcastle algorithm; a University of Louisville algorithm; an Uppsala University, Uppsala Sweden algorithm; a National University Hospital of Singapore algorithm; and a University of California, San Francisco algorithm. By selecting a desired algorithm form a drop-down menu, a physician using the electronic document 400 for Warfarin dosing may compare results from different algorithms and gain a better understanding of the range of dosages predicted.

With such an electronic document 400, a physician may enter any known demographic data and select a desired algorithm to calculate an estimated dosage for a treatment. The electronic document may then offer the option of printing out a hardcopy of the fully filled-out form and the resulting dosage, perhaps for inclusion in a patient's bedside chart. The electronic document may further include additional information to help facilitate use. Such additional information may include an abstracted patient identification number or test requisition number shown in human-readable form in item 415, or in machine scan-able barcode form in item 410. Also, a machine scan-able form of the embedded genotype data may be conveniently included in the form of a 2D barcode as in item 460. This latter item facilitates the creation of a new electronic document 400 from old paper printouts, should it ever prove necessary to do so. In addition to the ability to be printed out, the electronic document may have the ability to directly store historical data on disk or in a database. Such historical data may include a history of calculated dosages for treatments, dates of computation, demographic data used, and a specific algorithm used. Finally, the electronic document 400 may further include encryption and/or password protection as well as a set of instructions and/or directions for use as well as other caveats and warnings.

The nature of an electronic document 400 lends itself to a number of known and accepted document formats. The electronic document of FIG. 4 may comprise a portable document format, an extensible mark-up language format, a hypertext mark-up language format, compound document format, open document format or even a more conventional program written in a standard language such as Java or C++. In essence, the electronic document may be any computer-readable medium having computer-executable instructions wherein the various features and aspects of the electronic document 400 described above may be realized.

Further, the nature of an electronic document also lends itself to a high level of versatility when it comes to portability and duplication. Thus, any generated electronic document 400 may be delivered to a remote location in a number of manners including an electronic document attached to an electronic mail, an electronic document saved on a CD-ROM, an electronic document saved on a portable hard drive, an electronic document saved on a portable thumb drive, and an electronic document downloadable from a file transfer protocol network location. It will be appreciated that the nature of an electronic documents allows for several other manners of manipulation, duplication, generation and communication. Various examples of some methods for taking advantage of such a versatile electronic document 400 are described in the following paragraphs with respect to FIGS. 5 and 6.

FIG. 5 is a flowchart of a method for generating an electronic document 400 for determining a dosage for a treatment according to an embodiment of an invention disclosed herein. By way of overview in this example, a method for diagnosing a patient for a dosage of a treatment, includes receiving a genetic sample from a patient, identifying genetic characteristics of the genetic sample corresponding to the presence of one or more known gene sequences, generating an electronic document having the identified genetic characteristics embedded therein, the electronic document operable to calculate a dosage for a treatment based on the embedded genetic characteristics and demographic data about the patient.

Delving into the details of the methods shown in FIG. 5, the exemplary method starts at step 500 and proceeds to step 510 where specific genetic samples are collected from a source, e.g., blood from a patient, or the like. Based upon the nature of the dosage estimate being sought, specific gene markers may be identified from the genetic sample at step 512. As genetic data is gleaned from the genetic sample, it may be stored in a database of genetic information along with abstracted demographic information about the source of the genetic sample at step 514. Such a database may be utilized to assist in facilitating the generation and use of an electronic document 400 as described above with respect to FIG. 4.

At step 516, an electronic document may be generated based on gleaned information from the database. If an electronic document is to be created, the method moves to step 520 where a specific kind of electronic document is selected as detailed further in FIG. 6. In this example, a Warfarin dosage electronic document may be selected in a portable document format. As a particular electronic document is assembled, additional data may then be added wherein abstracted patient data, abstracted physician data, and encryption is added before the electronic document is ready for delivery, use, and/or deployment.

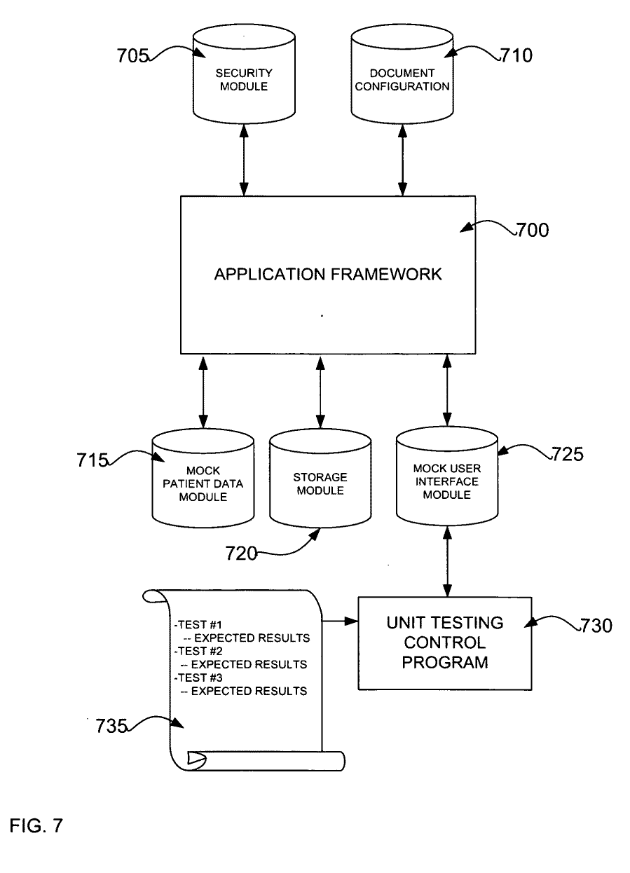

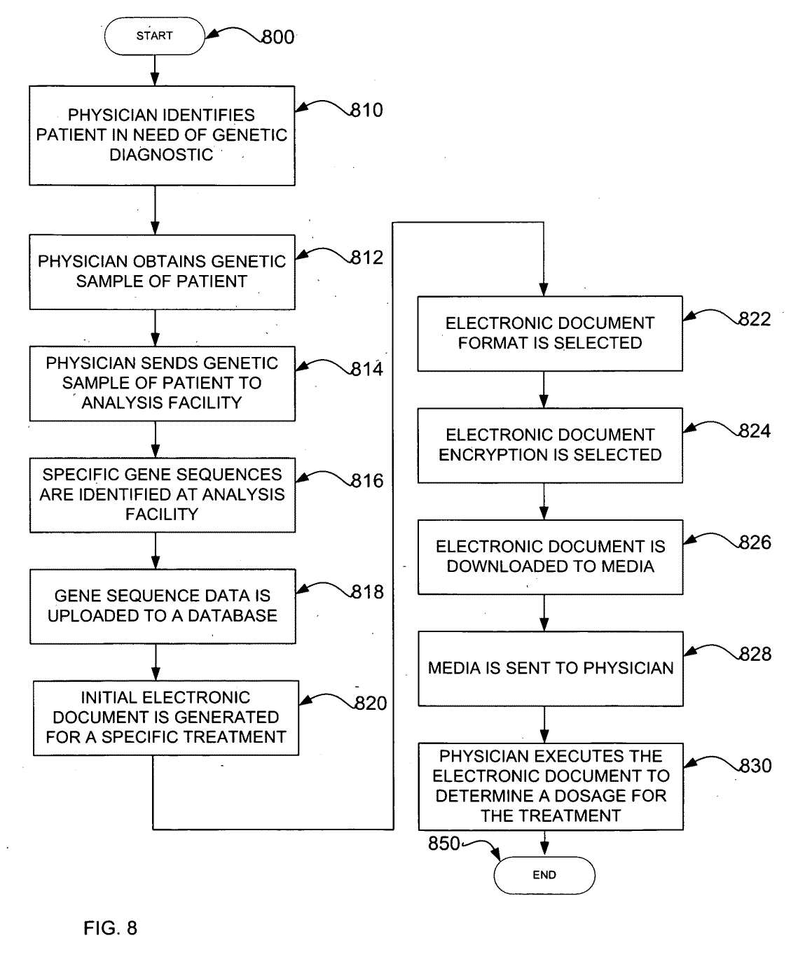

The method may return to decision step 516 and additional electronic documents may be created by repeating the process. If there are no more documents to create, then the method ends at step 590. Such an electronic document creation method may be practiced in a larger context of a genetic diagnostic delivery system as described in the method shown in FIG. 8. FIGS. 6 and 7 provide additional details as to the generation of such an electronic document.

FIG. 6 is a flowchart of a particular method for realizing an electronic document for determining a dosage for a treatment according to an embodiment of an invention disclosed herein. In one embodiment, the electronic document is implemented in Adobe Systems™ PDF (Portable Document Format). This form is particularly useful in that this execution environment is freely available and essentially ubiquitous in modern computer systems. PDF is also well-regarded for producing aesthetically pleasing documents that print identically on a wide variety of printers. In this case, the executable language is JavaScript, a dynamically-typed language widely used to provide client-side computation in web pages. Typically this kind of document can be constructed programmatically by means of one of the many available PDF generation libraries such as iText.

As illustrated in FIG. 6, the main scheme of the document generation process comprises a number of program steps, which may be implemented using any of the various programming languages known to those skilled in the art, such as the Java language. The process begins with selection of a sample ID to process at step 610, which may read from a file, selected from a database or solicited as user input via some type of user interface.

Next, the sample ID is used to retrieve the genotype data corresponding to that ID at step 612. As before, such data may be obtained by parsing the contents of a file, possibly even the same file from which the sample ID was extracted, selected from a database, queried from a networked measurement instrument or otherwise procured from any of the various electronic data sources known to those skilled in the art. Optionally, at step 614, delivery and patient contact information may also be obtained as well, such as the recipient's name, date-of-birth or Social Security Number. Additionally an email address or a password with which to encrypt the document may need to be obtained, the latter to ensure sensitive patient information is not compromising whilst the document is in transit to its ultimate recipient.

With the requisite information now in hand, the appropriate report to generate now may be determined at step 616. Typically this may be inferred from the scope of the genotype data at hand. It should be noted, however, that many pharmaceuticals are metabolized by the same enzymatic pathways, and so there may be considerable overlap in the set of alleles for different electronic documents used in different treatments. Therefore, other sources of information may have to be consulted to ensure the correct document is selected. The sample ID itself may prove useful for this purpose.

At step 618, report generation may commence. The program accomplishing this may be a subroutine of the main sample ID processing program or may be an entirely separate program which is invoked programmatically. In the case of a Java main program, a report generation script written in the Groovy language is particularly useful in that it may be run within the same JVM as the main program, and may therefore share in-memory data structures. This architecture also permits cosmetic changes in the final report format to be made more easily in that the main program need not be recompiled to effect the change.

Generation of a PDF file can be easily achieved through the use of a dedicated PDF manipulation library such as iText. This library provides an easy to use API for all steps of document creation and modification from a Java or Groovy environment. Typical steps in the creation process are shown in FIG. 6. These include: creating the document; setting access permissions; setting passwords and selecting encryption schemes; defining the overall document layout; creating input devices such as textfields, checkboxes and pulldowns; injecting genotype data; inserting executable JavaScript; generating and positioning printable barcodes and finally rendering and closing the document.

At this point the PDF document is essentially complete. At step 620, the method queries as to whether to document is generated. Additional steps can include such things as human examination of the document, checking resultant dosages for known input data, and finally applying digital signatures to certify the document meets all appropriate standards. If an error is detected, it may reported at step 630. If the document has been generated correctly, it may be stored or transmitted at step 632 before the method ends at step 640.

In further aspects of the various embodiments, the electronic document may be compiled in conventional fashion from source code in any of the programming languages known to one skilled in the art. As is well known in the art, much of the source code is unchanging and common to all documents of a given type. A portion of the source code, however, is to be generated automatically from the subject's genotype data. This can be done fairly simply, for example by using the data to define constants in the form of character strings, numeric values in arrays or the like. This synthetic source code may then be merged with the main body of code and then compiled to derive the finished electronic document.

In yet further embodiments, use of the programming technique known as inversion of control may be implemented. In this imperative style of programming, certain well defined and standardized operations are needed to render the correct op-codes comprising a PDF file. These operations are provided as subroutines in a PDF generation library, e.g. the iText library, which are invoked by the main program. The order of the invocation of these subroutines and thus the layout and behavior of the resulting PDF document is controlled by the structure of the main program. To change the document's behavior, one typically changes the main program, although in this, the burden may be lightened somewhat by abstracting this into a document generation script which is read by the main program.

Now consider this approach, which employs the technique of inverted control. The behavior required of an interactive document is a fairly standard type, namely responding to a sequence of events instigated by the human user of the document. Examples of this would be responding to key presses and releases, mouse motions and mouse clicks. This is, thus, a specific type of inverted control known in the art as event-driven programming and many application frameworks exist for providing this type of program architecture. Whereas in one case, the custom code invoked the general purpose library, but in a second case, the general purpose library invoked the custom code. This architecture lends itself well to such an electronic document generation system as described herein, wherein the organism-specific data, namely the genotype and optional contact information may be converted to a patient data module. This module then is implanted into the partially finished document where it may be invoked when needed by the application framework. Techniques for accomplishing this are known as dependency injection and are well-known in the art. Other modules, such as the document configuration, security, storage and user interface modules are also contemplated, but not described in further detail for brevity.

FIG. 7 is a diagram of a system for testing an electronic document for determining a dosage for a treatment as generated by the method of FIGS. 5 and 6 according to an embodiment of an invention disclosed herein. Here, existence of a well-contained user interface module provides a particularly valuable refinement. In FIG. 7 a system is shown with an application framework 700 for testing electronic documents that have been generated through methods described previously and in conjunction with a storage module 720 a security module 705 and a documents configuration module 710. In this case, the normal patient data and user interface modules have been replaced with special replacement modules to facilitate automated testing of the electronic document. The mock patient data module 715 represents a particular genotype for which the resultant dosage is known. The mock user interface module 725 is so constructed as to simulate the events generated by the actions of a human user under the control of an external unit testing control program. The unit test control program 730 is in turn driven by a suitable script 735 describing the tests to be performed and the expected resulting dosages for comparison with the output of the electronic document. Any discrepancies may thus be quickly detected and the cause of the error investigated. This refined embodiment therefore provides an extra measure of safety which is essential to any modern medical instrument.

FIG. 8 is a flowchart of a method for diagnosing a patient for a dosage of a treatment using an electronic document having the patient's genetic information and demographic information according to an embodiment of an invention disclosed herein. The method begins at step 800 and proceeds to step 810 wherein a physician may identify a patient in need of genetic diagnostics in order to more accurately prescribe a dosage for a treatment. As is the case with the running example herein, the dosage sought may be a dosage for Warfarin needed to treat various blood clotting conditions. Next, the physician obtains a genetic sample from the patient; this is typically a blood sample, although any sample having genetic material (e.g. skin, other tissue or biological fluid containing cells.) will suffice.

Having obtained a genetic sample, the physician may then send the genetic sample to an analysis facility. Such an analysis facility may be a remote location or may be within the same location as the patient that is receiving treatment. If remote, the genetic sample may be received via a carrier service and the analysis facility may also be a location where the generating of the electronic document occurs. Any combination of facilities is contemplated as within the scope of a multi-faceted computing and analytic network as described above with respect to FIG. 3.

At step 816, the genetic material is analyzed and specific gene marker or gene sequences are identified. Such genetic data gleaned from the genetic sample is then assimilated into a database at step 818 such that additional associations and correlations may be drawn from the assimilated data. Having a database of genetic information (which is associated with abstracted patient identification data), an electronic document for determining a dosage for a treatment may be generated at step 820. The generation of this electronic document is described above with respect to FIG. 5.

At step 822, the electronic document is formatted into a specific and convenient format. Such known formats include portable document format, an extensible mark-up language format, a hypertext mark-up language format, compound document format, and open document format. In essence, the electronic document may be any computer-readable medium having computer-executable instructions wherein the various features and aspects of the electronic document described above may be realized. At step 624, encryption may be added for security reasons. Those skilled in the art will appreciate any number of electronic document encryption and security methods.

Next, at step 826, once the electronic document is completed, it may be downloaded to some form of media. For example, the electronic documents may be copied to a portable thumb drive, such that the portable thumb drive is then sent to the physician at the physician's location at step 828. The physician, now having the personalized electronic document with a genetic diagnostic tool unique to the patient may execute various algorithm and calculations available on the constructed electronic document. For example, the physician may input demographic data into the generated electronic document and engage a calculation function to calculate the dosage for the treatment. Further, the physician may input a second set of demographic data into the generated electronic document and engage the calculation function to calculate a second dosage for the treatment. Essentially, the electronic will always yield an accurate and up-to-date dosage because of the dynamic nature of repeatable inputs of demographic data. The method then ends at step 850.

Various permutations of this method are contemplated. For example, in one embodiment, the genetic sample may be received and analyzed for specific genetic characteristics at a first location, while then sending data corresponding to the identified genetic characteristics to a second location (in an electronic format) for generating the electronic document at the second location. As another example embodiment, the generated electronic document may then be delivered to a remote location, in the form of an electronic document attached to an electronic mail, an electronic document saved on a CD-ROM, an electronic document saved on a portable hard drive, an electronic document saved on a portable thumb drive, or an electronic document downloadable from a file transfer protocol network location.

While the subject matter discussed herein is susceptible to various modifications and alternative constructions, certain illustrated embodiments thereof are shown in the drawings and have been described above in detail. It should be understood, however, that there is no intention to limit the claims to the specific forms disclosed, but on the contrary, the intention is to cover all modifications, alternative constructions, and equivalents falling within the spirit and scope of the claims.

What is claimed is:

1. A method for diagnosing a patient for a dosage of a treatment, the method comprising:
receiving a genetic sample from a patient;
identifying genetic characteristics of the genetic sample corresponding to the presence of one or more known gene sequences;
generating an electronic document having the identified genetic characteristics embedded therein, the electronic document operable to calculate a dosage for a treatment based on the embedded genetic characteristics and demographic data about the patient.

2. The method of claim 1, further comprising generating the electronic document with encryption.

3. The method of claim 1, further comprising generating the electronic document in a format from a group comprising: a portable document format, an extensible mark-up language format, a hypertext mark-up language format, a rich-text format, compound document format, and open document format.

4. The method of claim 1, further comprising generating the electronic document having abstracted data corresponding to an identification of the patient.

5. The method of claim 1, further comprising generating the electronic document having abstracted data corresponding to an identification of a doctor associated with the patient.

6. The method of claim 1, further comprising receiving the genetic sample via a carrier service at a location where the identifying of the genetic characteristics and the generating of the electronic document occurs.

7. The method of claim 1, further comprising:
receiving the genetic sample and identifying of the genetic characteristics at a first location;
sending data corresponding to the identified genetic characteristics to a second location in an electronic format; and
generating the electronic document at the second location.

8. The method of claim 1, further comprising delivering the generated electronic document to a remote location, the delivery comprising one of a group comprising: an electronic document attached to an electronic mail, an electronic document saved on a CD-ROM, an electronic document saved on a portable hard drive, an electronic document saved on a portable thumb drive, and an electronic document downloadable from a file transfer protocol network location.

9. The method of claim 1, further comprising:
inputting demographic data into the generated electronic document; and
engaging a calculation function to calculate the dosage for the treatment.

10. The method of claim 9, further comprising:
inputting a second set of demographic data into the generated electronic document; and
engaging a calculation function to calculate a second dosage for the treatment.

11. The method of claim 9, wherein the inputted demographic data comprises demographic data from a group comprising: age, weight, height, gender, ethnicity, propensity to smoke, and genotype data.

12. A system for diagnosing a patient for a dosage of a treatment, the system comprising:
a means for receiving a genetic sample from a patient;
a means for identifying genetic characteristics of the genetic sample corresponding to the presence of one or more known gene sequences;
a means for generating an electronic document having the identified genetic characteristics embedded therein, the electronic document operable to calculate a dosage for a treatment based on the embedded genetic characteristics and demographic data about the patient.

13. The system of claim 12, further comprising a means for formatting the electronic document into a format from the group comprising: a portable document format, an extensible mark-up language format, a hypertext mark-up language format, compound document format, and open document format.

14. The system of claim 12, further comprising a means for embedding patient and physician identification information into the electronic document in an abstracted manner.

15. A system for calculating a dosage of a treatment using a personalized diagnostic electronic document, the system comprising:
a genetic sample collection system operable to collect a sample of genetic material from a patient;
a genetic sample analysis system operable to receive the sample of genetic material and operable to identify the presence of at least one of a plurality of analytes; and
a client computer operable to generate an electronic document, the electronic document comprising:
embedded data representing the presence of at least one of a plurality of gene markers;
at least one input field operable to receive an input corresponding to demographic data a source of the genetic material; and
a computational field operable to generate a dosage of a treatment based on the embedded genetic data and the inputted demographic data.

16. The system of claim 15 wherein the dosage for a treatment comprises a dosage for Warfarin.

17. The system of claim 15 wherein the at least one of a plurality of gene markers comprises an analyte from a group comprising: CYP 2C9*2, CYP 2C9*3, CYP 2C9*4, CYP 2C9*5, CYP 2C9*6, CYP 2C9*11, VKR3673, VKR5808, VKR6009, VKR6484, VKR6853, VKR7566, VKR8773, and VKR9041.

18. The system of claim 15 wherein the demographic data comprises at least one of a group comprising: use of delavirdine, use of fluvoxamine, use of nifedipine, use of phenylbutozone, use of fluconazole, use of loratodine, use of omeprazol, use of fluvastatin, use of nicardipine, use of paroxetine, use of tobacco, gender, age, weight, height, and ethnicity.

19. The system of claim 15 wherein the calculation is based upon a specific Warfarin treatment algorithm, the Warfarin treatment algorithm selected from a group comprising: a University of Newcastle algorithm, a University of Louisville algorithm, a National University Hospital of Singapore algorithm, and a University of California, San Francisco algorithm.

20. The system of claim 15 further comprising a database of identification information coupled to the client computer, the database comprising abstracted information regarding the identification of a patient and the identification of a physician associated with the patient.
